(19) United States
(12) Patent Application Publication
(10) Pub. No.: US 2013/0045535 A1
(43) Pub. Date: Feb. 21, 2013

(54) NICHE SYSTEM FOR BIOLOGICAL CULTURING

(71) Applicants: Yoav Soen, Rehovot (IL); Saar Golan, Rehovot (IL)

(72) Inventors: Yoav Soen, Rehovot (IL); Saar Golan, Rehovot (IL)

(73) Assignee: Yeda Research and Development Co. Ltd. at the Weizmann Institute of Science, Rehovot (IL)

(21) Appl. No.: 13/695,139

(22) PCT Filed: Apr. 26, 2011
(86) PCT No.: PCT/IL11/00333

§ 371 Date: Oct. 29, 2012

Related U.S. Application Data
(60) Provisional application No. 61/328,249, filed on Apr. 27, 2010.

(51) Int. Cl. C12M 3/00; B29C 69/02; C12N 5/071

(52) U.S. Cl. 435/395; 435/289.1; 435/325; 264/250

(57) ABSTRACT

A cell culturing system is provided. The cell culturing system includes a cell niche defined by a niche base and niche walls, at least one of which includes a fluid pathway formed therein. The niche wall material is selected capable of enabling diffusion into the niche of a fluid flowing between an inlet and an outlet of the fluid pathway.

### FIELD AND BACKGROUND OF THE INVENTION

The present invention relates to a culturing niche system and methods of making and using same. Embodiments of the present invention relate to a 3D niche system and uses thereof in tissue engineering, as well as culturing and studying the development and differentiation of cells (e.g., stem cells and progenitors) and cell structures (e.g., EBs and embryos). Further embodiments of the invention relate to the study of cancer stem cells and tumors, tumorogenesis and metastasis (e.g., anti-cancer drug testing).

Embryonic and adult stem cells of human origin provide unique models for studying human development and at the same time, exciting potential sources of cells for regenerative medicine. Efficient realization of this potential, however, requires in vitro models supporting the development of human embryonic tissues and allowing mass production of therapeutically useful cells. Of particular importance is the developmental and therapeutic potential of human embryonic stem cells (hESCs). When induced to differentiate, hESCs can give rise to any cell type of the embryo proper, as well as extra-embryonic tissues [Thomson et al. 1998]. These cells are currently grown and induced to differentiate in a dish under uniform and relatively static conditions. Nevertheless, in vivo, embryonic and adult stem cells grow and function within three dimensional (3D) niches exhibiting complicated spatial and cellular structure which typically reflects the developmental stage of the organism and its current physiological demands [Li and Xie 2005]. The niche (or a micro-environment) is a basic unit of tissue physiology that supports stem cells and regulates their participation in tissue generation, maintenance and repair [Scadden 2006, Saha et al. 2007]. The ability to mimic the structure and function of such niches and to recapitulate embryonic-like self-organization in vitro can lead to breakthroughs in medical practice and in basic research. Unfortunately, this goal can not be accomplished using standard tissue culture techniques—i.e., using conventional dishes where the exposure to biochemical signals is uniform and does not support spatio-temporal patterning. For example, a key feature that is missing from current techniques is the ability to control the local supply of nutrients, hormones and morphogens which participate in tissue patterning. Recent developments in microfluidic scaffold design can partially address this significant shortcoming with respect to individual cells [Choi et al 2007]. However, culture of extended embryonic structures and embryos requires a design that takes into account larger size scales and controls the supply patterns of nutrients, growth factors, and soluble gases across the developing tissue. Optimal design will also facilitate time lapse confocal microscopy, long term incubation on the microscope stage, online collection of metabolites produced during the culturing period, and retrieval of samples for further analysis (e.g., immunohistochemistry). Culturing systems of this kind are currently lacking. Present techniques of stem cell growth and investigation are based almost invariably on 2D culture in a dish or simple bioreactors optimized for up-scaling cell numbers [King and Miller 2007]. Attempts to construct sophisticated (typically microfluidics-based) devices to control the micro-environment are yet limited to very few cells [Choi et al. 2007] or static [Soen et al. 2006] and typically lack either the third dimension or dynamic control over the micro-environment. Consequently, these approaches are less compatible with the concept of a niche which supports the growth and dynamic patterning of extended embryonic tissues in a constrained 3D region.

Cancer remains a key mortality factor (worldwide) for the past several decades. Mortality rates in the U.S. range between 100 and 300 deaths per year per 100000 people (covering changes in race, sex and ethnicity) [SEER Cancer statistics review for the years 1975-2007, FIG. 2.4]. Furthermore, the overall mortality rates have hardly changed over the past 32 years. This data suggests that radically novel methods to explore cancer development and design anti-cancer drugs are required.

### SUMMARY OF THE INVENTION

According to one aspect of the present invention there is provided a cell culturing system comprising at least one cell niche defined by a niche base and niche walls, wherein at least one niche wall of the niche walls includes a fluid pathway formed within the niche wall and further wherein a material of the niche wall is selected capable of enabling diffusion into the at least one cell niche of a fluid flowing between an inlet and an outlet of the fluid pathway.

According to further features in preferred embodiments of the invention described below, the material is a biopolymer gel.

According to still further features in the described preferred embodiments the niche walls angle out from the niche base.

According to still further features in the described preferred embodiments the cell culturing system further comprises a support scaffold for supporting at least one cell niche.

According to still further features in the described preferred embodiments the cell culturing system further comprises a biodegradable scaffold contained within the cell niche.

According to still further features in the described preferred embodiments the cell culturing system further comprises biomolecules and/or cells trapped within or attached to the biodegradable scaffold.

According to still further features in the described preferred embodiments the material enables diffusion of molecules carried by the fluid.

According to still further features in the described preferred embodiments the material is impermeable to diffusion of cells.

According to still further features in the described preferred embodiments the cell culturing system further comprises a chamber for housing the support scaffold.

According to still further features in the described preferred embodiments the chamber includes a bottom cover slip mountable on a microscope stage.

According to another aspect of the present invention there is provided a method of fabricating a cell culturing chamber comprising: (a) 3D printing a housing of the cell culturing chamber, the housing having inlet and outlet ports and a support scaffold for containing at least one cell culturing niche; and (b) casting the at least cell culturing niche in the support scaffold.

According to still further features in the described preferred embodiments the housing includes an internal component threadable into an external component.

According to still further features in the described preferred embodiments the at least one cell culturing niche is cast from a biopolymer gel.

According to still further features in the described preferred embodiments the cell culturing niche is cast over at least one wire positioned between the inlet and the outlet ports, the wire being removed following casting to form a fluid pathway in a wall of the at least one culturing niche.

According to still further features in the described preferred embodiments the housing further comprises top and bottom cover slips.

According to yet another aspect of the present invention there is provided a method of culturing cells or cell structures comprising seeding cells or cell structures into the culturing system described above.

According to still further features in the described preferred embodiments the cells or cell structures are seeded with a biodegradable scaffold.

The present invention successfully addresses the shortcomings of the presently known configurations by providing a cell culturing niche and system capable of more closely mimicking the anatomic and biochemical environment of in-vivo niches.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-F illustrate one embodiment of the ex vivo niche of the present invention. FIG. 1A—schematics of the ex vivo chamber. The center niche is exposed to a gradient of biochemical component (e.g., protein) created by diffusion from the fluid pathways (also referred to herein as ‘capillaries’) at its margins. Each of the outer niches (control niches 1 & 2) is relatively uniformly exposed to the component concentration at its neighboring fluid pathway. FIG. 1B—diagram of the experimental setup. FIG. 1C—top: exemplary profiles of substance concentration across the niche, demonstrating the ability of the design to independently control the gradient and the concentration; bottom: simulation of the diffusion process showing the resulting steady state concentration of a substance that is supplied by one of the fluid pathways. FIG. 1D—top view of the fabricated device connected to external lines of differentially colored dye tracers. The magnified region shows the gradient at the central niche and the uniform content at the control outer niches. FIG. 1E—cross section of the niche region in an actual device. FIG. 1F—left: PDMS dish containing multiple micro-wells for production of embryoid bodies (hEBs) of a defined and uniform size. The hEBs are used as a model of early human embryos. They are formed by centrifugal aggregation of a suspension of human embryonic stem cells (hESCs) into the micro-wells. Right: hEBs forming at the micro-wells.

FIGS. 2A-S illustrate the process of diffusion from the fluid pathways into the niche. FIG. 2A schematically illustrates diffusion in the chamber. The source (fluid pathway (‘capillary’) is represented by the filled circle #1 and the sink capillary by the hollow circle #4. Source and sink reflections #2,3,7 and #5,6,8, respectively, are positioned properly so as to represent the zero flux boundary conditions. FIG. 2B shows a surface plot of the concentration distribution in the chamber. FIG. 2C shows the concentration profile along the chamber width at several height planes. FIG. 2D shows the geometric model of the chamber for the numeric model. FIG. 2E shows the employed finite element mesh. FIG. 2F shows the solute distribution in the chamber following the onset of perfusion. FIG. 2G shows a representative transient solute distribution during the diffusion process. FIG. 2H shows the solute distribution after stabilization of the diffusion process. FIG. 2I shows the solute profile along the chamber width at the bottom plane at various time points. FIG. 2J shows the solute profile along the chamber width at the capillary plane at various time points. The capillaries are absent from the image as they participate in the model only as boundary conditions. FIG. 2K shows the solute profile along the chamber width at the top plane at various time points. FIG. 2L illustrates a mouse embryo cultured within a soft agar niche (transmitted light). FIG. 2M is a fluorescence image corresponding to the inset in FIG. 2L (The embryo is dyed using a Dil membranal dye, Biotium, see Pawley 1995). FIGS. 2N-P are top views of the device with no patterned niche connected to external lines of differentially colored dye tracers (50 μl/hr flow) after 30 minutes, 3 hours and 1 day, respectively. The diffusion front of the two fluids is clearly visible. FIG. 2Q—same as FIG. 2P but with one patterned niche. FIG. 2R—calcein profile along the chamber width at the capillary plane at various time points. A simple moving average (SMA) data smoothing algorithm (fifty element) is used filter the noise. FIG. 2S—same as FIG. 2R but for the GFP-Coh2 protein.

FIGS. 3A-D are isometric (FIG. 3A), top (FIG. 3B), front (FIG. 3C) and exploded (FIG. 3D) views of the present system.

FIGS. 4A-D demonstrate recapitulating implantation using embryos and primary endometrial cells using the present system. FIG. 4A—a Bovine blastocyst was embedded in a biodegradable scaffold (Alginate) supplemented with bovine endometrial cells into a primitive niche lacking the fluid pathways. Shown is a vasculature-like network of cells that is formed only when the blastocyst and the endometrium are co-cultured. Left inset shows a magnified field in transmitted light (TL), DAPI staining and an overlay. FIG. 4B—Same as A but with a mouse blastocyst and mouse endometrial cells. FIG. 4C—an embryoid body implantation recapitulation experiment. Top: An EB consisting of trophoblasts (JAR, labeled NLS-mCherry) and ESCs (H1, labeled CFP) initiating attachment to a layer of receptive endometrium (ECC-1, labeled GFP). Bottom: Same as top but after three days of co-culturing. The trophoblasts have completely invaded/displaced the endometrial layer and attached to the plate bottom. FIG. 4D—an embryoid body developmental plasticity experiment. An EB containing ESCs (H1) constitutively labeled with CFP and OCT4-GFP knock-in cells (H1, courtesy of the Thomson Lab) was cultured at the niche. Differentiation media (based on BMP-4) was perfused at one capillary and pluripotency maintaining conditioned media (from mouse embryonic fibroblasts, based on bFGF) was perfused at the other. The time-lapse image series (here a set of four images with one hour interval) demonstrates differentiation/morphogenesis processes occurring within the EB. The EB exhibits internal structures (see red arrows) and experiences localization of OCT4-GFP pluripotent cells. It also presents an extrusion that was not present in the original spherical structure (a phenomenon repeatedly observed in additional experiments).

FIG. 5 illustrates a niche array for high throughput experimentation/industrial screening.

### DESCRIPTION OF THE PREFERRED EMBODIMENTS

The present invention is of a cell culturing system which can be used to culture and study stem cells, embryoid bodies (EBS) and embryos.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

3D culturing systems designed for use as stem cell niches are known in the art. Such systems are designed for replicating the environment of natural anatomical niches but utilize fluid/nutrient delivery approaches which result in suboptimal supply of nutrients, growth factors, and soluble gases to the developing tissue.

While reducing the present invention to practice, the present inventors have devised a culturing system that utilizes one or more cell niches which are configured for enabling capillary-like diffusion of fluids and nutrients through the niche wall and to cells/tissue grown within the niche. Due to its unique fluid/nutrient delivery design, the present system provides an infrastructure for a broad range of applications in tissue engineering, and also provides a unique model for studying aspects of morphogenesis such as blastocyst implantation, gastrulation and post-gastrulation development that were not previously accessible for in vitro investigation.

Thus, according to one aspect of the present invention there is provided a cell culturing system which includes at least one cell niche.

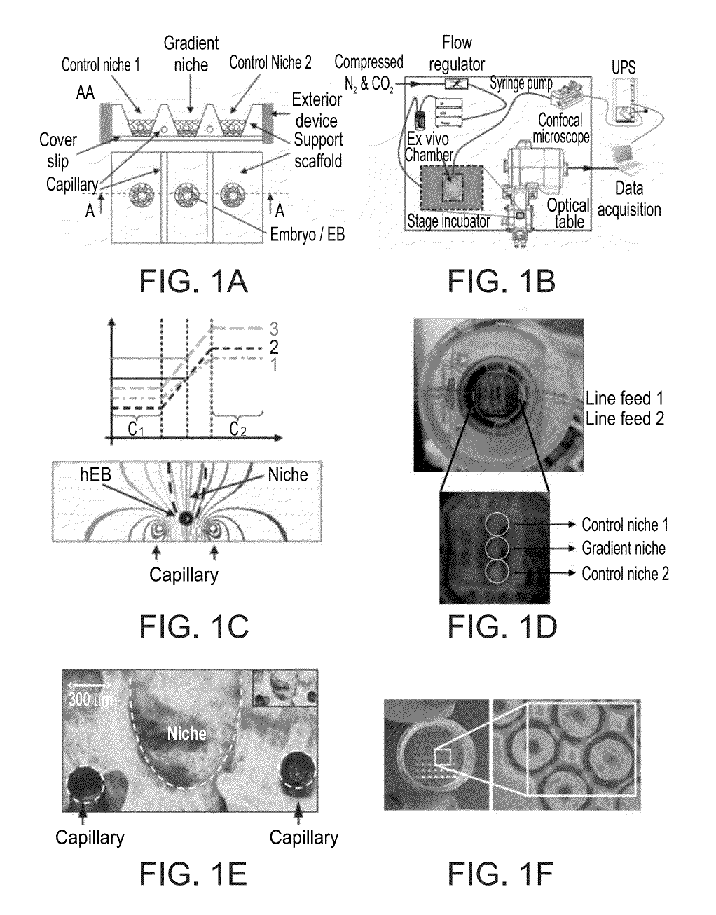

The cell niche includes a base and walls which define a volume (typically 10-30 μl). The walls are preferably angled out from the base to form a substantially V-shaped niche (see FIG. 1A). Such a niche shape is advantageous since it enables variable height culturing of cells/structures of varying radii (up to 900 μm) and thus provides sample size accommodation. A cell having a radius of about 10 μm can be cultured at a first height, whereas a cell structure (e.g. EB) having a radius of 500 μm can be cultured at a second (and higher) height within the niche.

As is further described hereinunder, the niche walls are constructed from a fluid permeable material (e.g. biopolymer gel such as agarose) which includes fluid pathways within at least one wall along a length thereof (see FIG. 1A). As is further described hereinunder, such pathways can be formed by casting the niche over wires and pulling the wires out of the cast niche material after solidification.

The present system also includes a support structure (also referred to herein as a support scaffold) which enables niche generation (casting) and supports formed niches while providing access to fluid inlet and outlet connections. The niche and support structure/scaffold are collectively referred to herein as a cell chamber.

The fluid pathways include inlet and outlet ends which are disposed at the ends of the chamber wall. Fluid flowing between the inlet and outlet ends diffuses through the fluid pathway walls (formed from the niche wall) and into the niche itself. Thus, by enabling diffusion of fluid (and the fluid carried molecules) along the entire length of the niche wall, the fluid pathway(s) functions similarly to capillaries that line anatomical niches. This provides a more efficient way to deliver fluid and nutrients as well as control the concentration of nutrients the tissue grown in the niche is exposed to. The inlet and outlet ports can also be used for monitoring and collecting samples of ions, molecules and gasses excreted/produced by the biological sample, to thereby track the state (e.g. differentiation state) of the biological sample.

Components of the present culturing system can be fabricated using conventional approaches such as casting, CNC milling and the like.

A preferred approach for fabricating the system of the present invention employs computer-aided design (CAD) followed by fabrication using a 3D printer; this approach is favored due to the small volume of the cell niche (10-30 μl), its low cost, as well as its adaptability to mass production.

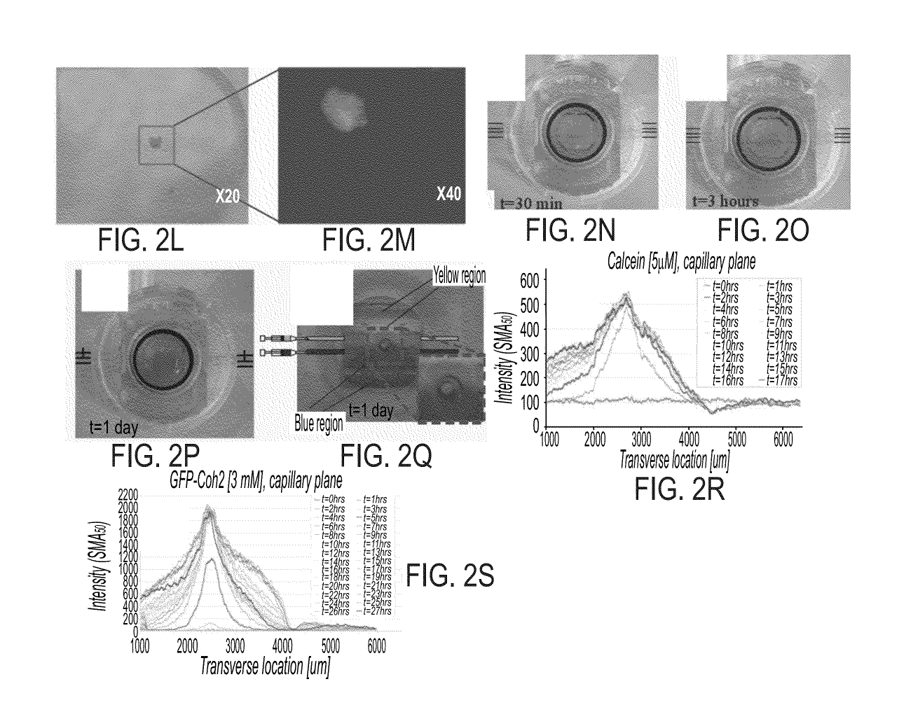

To facilitate robustness and repeatability, CAD files and 3D-printing (Eden260VTM, Objet Geometries), rather than fabrication drawings are used for fabricating components of the present system. This approach has several advantages. It minimizes improper manufacturing by removing human errors and ensures robust (repeatable) fabricated components. It also reduces the device price since the fabrication is entirely automated. However, most important, it enables mass production of the complicated components (e.g. the chamber components described hereinunder with reference to FIGS. 3A-D). Several dozen systems can be fabricated on a single tray within few hours in one printer work cycle. Furthermore, the fabrication can be performed simultaneously at multiple locations and by numerous manufacturers since the CAD files can be sent via e-mail to any 3D printing facility.

A presently preferred fabrication material is the FullCure 720 biocompatible and transparent material (objet), although other materials such as Titanium 64 can also be used for 3D printing (laser sintering) fabrication of the chamber components of the present system.

The chamber of the present system can include one or more niches which share one or more capillary-like fluid pathways and a single support structure (support scaffold).

FIGS. 1A-F illustrate a three niche, two fluid pathways embodiment of the present system. The niches accommodate the biological sample within a specified sub-region in between two perfusion/feeding fluid pathways that run inside a fluid-permeable niche wall. Such a diffusion configuration enables dynamic control over nutrients, signaling cues, and soluble gases in the niche. This design is unique by virtue of enabling independent and dynamic compound-specific control over the concentration and the gradient of each of the compounds that are delivered to the niche (FIG. 1C, top).

The niche and capillary dimensions are derived/determined from the dimensions of the biological ample studied—EBs, blastocysts and eggs that range between tens of microns and nearly 1 mm in diameter (e.g. Xenopus egg). To accommodate such a wide range, the niche is preferably V-shaped or conical with a cross section linearly increasing with its height (see FIG. 1E). It will be appreciated however, that other niche configurations (e.g. rectangular, square, semi-circular) are also envisaged herein. The niche base is adjacent to a first cover slip at the bottom of the cell chamber (standard 24 mm diameter, 170 μm thickness, #1.5 cover slip). This cover slip contacts the microscope objective. The preferred conical structure of the niche also facilitates cost-effective use of costly biodegradable scaffolds and ECM components. For example, when used for culturing stem cells, only a 20 μl volume of the niche tip is used.

The fluid pathways are positioned within the niche walls as close as possible to to the internal wall surface in order to obtain maximal gradient values per given perfused component concentration. Typical fluid pathway diameter is between 100-300 μm, preferably 200-300 μm. Reducing the diameter below 100 μm may impede flow rates and thus the characteristic time of the chamber wash by convection and is therefore less preferred. It is also desirable to maintain large enough source and drain capillaries so as to ensure dominant transverse diffusion patterns (see FIG. 1C, bottom). Furthermore, large source/drain capillaries ensure smaller characteristic times for the build up of component concentration/gradient by diffusion. It is important to note that chamber miniaturization is highly desired since smaller volume facilitates the use of less culture medium and less morphogens and growth factors for the concentration/gradient build up as well as faster convection/diffusion rates.

The chamber height should be large enough to accommodate the niches and capillaries but minimal due to miniaturization requirements similar to those described above. Furthermore, to obtain maximal working distance for the microscope objective it is necessary that the niche tip region containing the studied biological samples be as close as possible to the objective position at the bottom cover slip.

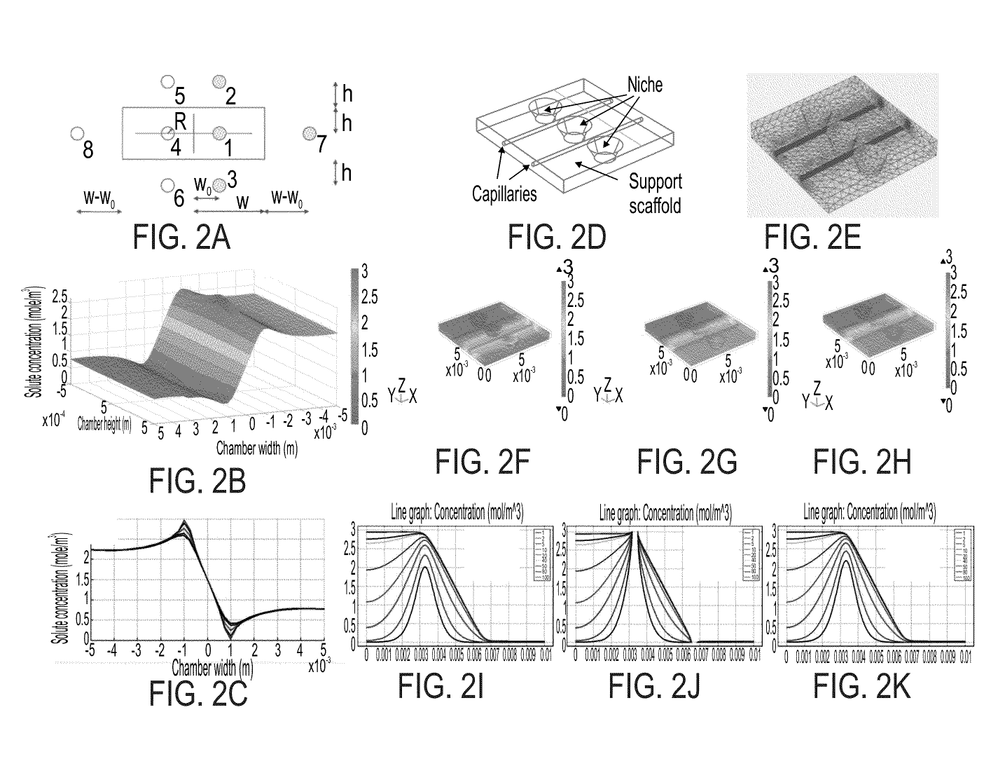

The biochemical makeup of the niche reservoir must be explicitly controlled at all times (concentrations and gradients). Therefore, a simple well design (such as in multi-well plates) will not suffice. It is essential to enable medium (protein) and small molecule (e.g., cytokine/morphogen/growth factor) motion into the niche while simultaneously restraining the cells and the developing tissue from leaving the niche. To comply with these demands the niches are fabricated from a fluid-permeable (porous), cell-impermeable material. This material should be bio-compatible with the cells/tissue and have pores that are much larger than those of small molecules/proteins but much smaller than cells. one preferred material is low melting agarose (SeaKem® LE agarose, 1% w/v, Lonza). The pore size of low melting agarose at 37° C. is above 1200 nm (Janaky et al., 2006). This material retains the cells/embryos within the niche while enabling small molecule transfer (FIGS. 1D and 2A-S).

FIGS. 1D and 2A-F also demonstrate that the number of niches (3 in this case) does not appreciably affect gradient formation and the kinetics of the diffusion process.

The niche can be fabricated using casting or milling techniques. One preferred approach employs a niche cavity filler element which is similar in function to a wen-forming comb used to form sample loading wells in an electrophoresis gel. The support scaffold is filled with a gelling biopolymer (e.g. agar) and an aligner jig is used to align the niche cavity filler element (with its three niches tips) with the support scaffold (see FIG. 3B). Once the agar sets (gels), the niche cavity filler element is removed and the niches are formed. The capillaries are formed in a similar manner by positioning two wires of 100-300 μm diameter between the two inlets and two outlets of the chamber and pulling them out following agar solidification. In this manner the agar forms the niches and the capillaries in a homogenous and isotropic material using a single casting step.

One of the key objectives of the present invention is supporting the culture of developing (self organizing) embryonic tissue under conditions as close as possible to those present in anatomical (in-vivo) niches. Therefore, it is necessary to culture the samples in a supportive 3D environment—which preferably requires the use of a scaffold.

An alginate based scaffold (Protanal LF 10/60, 2% w/v, FMC BioPolymer) and BD Basement Membrane Matrix (Matrigel™, stock) can be used to support ESC and embryo proliferation in the niche and are compatible with transmitted light and fluorescent microscopic observation thus allowing microscopic observation.

FIGS. 3A-D illustrate the components and assembly arrangement of one embodiment of the present system which is referred to hereinunder as system 10.

The footprint and height of system 10 were selected similar to a standard six cm dish. This ensures that system 10 is compatible with numerous microscope stage holders and particularly microscope on stage incubators. The volume of chamber 12 of system 10 is 1 ml. The biodegradable scaffold volume of each niche is roughly 20 μl. In this manner, the biodegradable scaffold used for culturing the biological samples is kept minimal. This is crucial since biodegradable scaffolds (e.g., collagen) are much more expansive than the support scaffold and constitute a financial limiting factor during experimentation. By limiting niche volume, system 10 enables the use of expensive culturing scaffolds such as stock Matrigel.

Chamber 12 of system 10 is assembled from two main components connected via, for example, a threaded lock. External component 16 (blue in FIG. 3A) constitutes the base of system 10. It houses lower cover slip 18 of chamber 12. lower cover slip 18 is fixed at its periphery by a circular groove/step 20 (formed in external component 16) and is held in place against an internal component 22 (yellow in FIG. 3A) which is threaded into external component 16. A fluid seal is obtained by using an O-ring 24 positioned within a groove 26 prefabricated in internal component 22. O-ring 24 also functions as an elastic buffer against concentrated mechanical forces that may break cover slip 18. After assembling external 16 and internal 22 components and O-ring 24 (e.g. standard no. 2-019), chamber 12 is tested for fluid sealing with 1 ml of 70% ethanol. In this manner, chamber 12 is also sterilized prior to introduction of biological material. The 70% ethanol is then aspirated and chamber 12 is washed twice with 1 ml PBS. 300 μm wires (not shown) and the niche cavity filler element 28 and aligner jig 30 are then positioned within support scaffold 32 and 1 ml of low melting agarose (SeaKem® LE agarose, 1% w/v at room temperature) is poured into support scaffold 32. This volume covers the wires and niche-forming tips of niche cavity filler element 28.

Upon solidification (˜10 min), the agar is washed twice with PBS (e.g. 0.7 ml) to remove excess agar. A culture medium is then added to support scaffold 32 to ensure that no air bubbles are trapped within niche cavities (not shown) and niche cavity filler element 28 and aligner jig 30 removed. The wires are then removed from chamber 12 (through capillary inlet shown in FIG. 3B). The niche cavities are then filled with a biodegradable scaffold (e.g., alginate or Matrigel) containing the cells/embryo.

Cell suspensions can be introduced directly to the niche. Embryos/hEBs are preferably embedded in the scaffold using a capillary microdispenser (e.g., Wiretrol—Drummond Scientific Catalog No. 5-000-1001) to ensure minimal damage and maximal survival rates of the embryos/EBs. Although there is no need to employ a microscope to dispense cell suspensions into the niche, embryo/EB transfer to the niche under microscopic/stereoscopic observation is preferred. After introducing the biological sample to the niches, a second O-ring 36 (standard no. 2-018) is positioned at a top groove 38 of internal component 22 and chamber 12 is sealed with a second cover slip 40 positioned on top of O-ring 36. The cover slip is then fixed in position using a threaded ring like cover 42 (brown in FIG. 3A). The assembled system can then be connected to fluid lines and initiated.

The present system was tested analytically, numerically and experimentally and the results are presented in FIGS. 2A-S and 4A-D.

At the steady state the diffusion equation reduces to Laplace's equation. The boundary conditions for the chamber configuration are zero flux on all walls, constant (positive) flux from the source capillary and similar (but negative) flux from the sink capillary. We solve this equation under the described boundary conditions by employing the superposition and method of reflections techniques (as in classic potential flow, assuming negligible axial changes) (Fundamentals of fluid mechanics, Munson, Young and Okiishi). The fluid pathways are modeled by a set of source (filled circle #1) and sink (hollow circle #4), FIG. 2A. The walls are represented by reflections from three sources (#2,3,7) and three sinks (#5,6,8) positioned outside the chamber borders. The obtained concentration field is:

$$C ( x , y ) = \frac{m}{2 π} [ ln {r}_{1} + ln {r}_{2} + ln {r}_{3} + ln {r}_{7} - ln {r}_{4} - ln {r}_{5} - ln {r}_{6} - ln {r}_{8} ]$$

where:

r₁²=(x−w₀)²+y²,r₂²=(x−w₀)²+(y−2h)²,r₃²=(x−w₀)²+(y+2h)²

r₄²=(x+w₀)²+y²,r₅²=(x+w₀)²+(y−2h)²,r₆²=(x+w₀)²+(y+2h)²

r₇²=(x−2w+w₀)²+y²,r₈²=(x+2w−w₀)²+y²

and where m is a parameter representing the source/sink intensity (the diffusive flux from/to the capillary per unit length):

$${j}_{r} ( r = R + ɛ , θ ) = - D \frac{\partial C}{\partial r} {\vert}_{r = R + ɛ} = - D \frac{m}{2 π R} , ɛ \to 0$$

This solution is graphically illustrated in FIGS. 2B-C (MATLAB). FIG. 2B presents a surface plot of the concentration distribution in the chamber. FIG. 2C presents the concentration profile along the chamber width at several height planes. It can be seen that the linear concentration gradient between the two capillaries is maintained quite well along the entire chamber height although its extreme values slightly decrease away from the capillaries plane. It is also evident that the control regions (to the left/right of the gradient region) are expected to be more uniform in concentration. The average concentration at the control regions is expected to be slightly less/more than at the source/sink, respectively.

The diffusion process kinetics was simulated using the finite element software COMSOL. Finite element is a numeric technique used for solving partial differential equations over complicated domains. It is based on discretization of the domain to small finite elements (e.g., triangles in 2D and tetrahedrons in 3D) and approximating the differential arguments with algebraic differences. The end result is a set of algebraic equations represented and solved using matrix mathematics techniques. FIG. 2D presents the geometric model solved (which includes the three niches). FIG. 2E presents the employed finite element mesh (tetrahedrons). FIGS. 2F-H show three transient stages of the solute distribution in the chamber. FIGS. 2I-K show the solute profile along the chamber width at the bottom, fluid pathway and top planes, respectively.

As is further described in Example 1 of the Examples section which follows, The present system maintains stable gradients over long periods of time for both small molecules and proteins. Small molecules are relevant in experiments involving inducible systems (e.g., Doxycycline—M_{w}=444.435 Da) and in experiments involving antibiotics (e.g., Geneticin/G418—M_{w}=692.70 Da), while proteins are relevant in experiments involving differentiation processes using morphogens such as Wnt-3a (M_{w}˜37.4 kDa), BMP-4 (M_{w}˜2×22 kDa) and Noggin (M_{w}˜32 kDa).

The present system was also used for culturing EBs as is described in the Examples section which follows.

The present invention can be used for growing stem cells, tissues, and embryos for research or therapeutic purposes.

Cells or cell structures which can be cultured using the present system include, but are not limited to, 293T/293, Hela, ISHIKAWA, ECC1, JAR, H1 ESCs, H9 ESCs, Hues ESCs, primary bovine/mouse endometrial cells and bovine/mouse embryos.

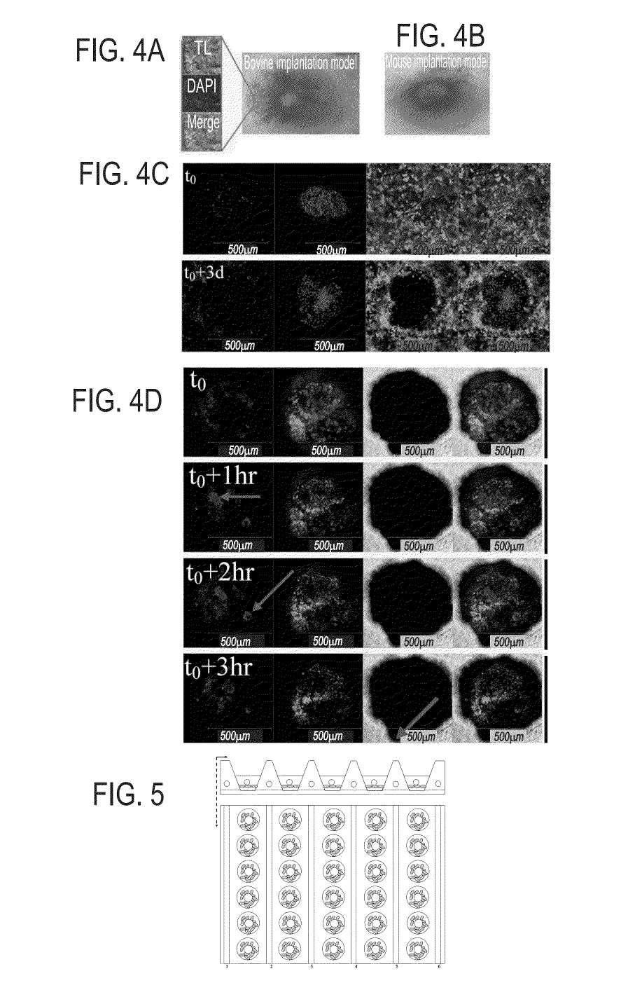

It will be appreciated that the present system is not limited to one or several niches but can include any number of niches (e.g. 10, 50, 100) under a single chamber enclosure. FIG. 5 illustrates a representative embodiment of a scaled up device for high throughput experimentation/industrial screening. The axial direction (dashed arrow) represents biological repetitions of the same experiment. The transverse direction (full arrow) represents varying experimental (flow) conditions. By modifying the fluid to flowing in each capillary (1-6 here) it is possible to explicitly control the concentration and gradient of each niche column Different columns can be entirely hydro dynamically buffered using neutral fluids (e.g., PBS).

Alternative configurations can include an additional set of capillaries that are orthogonal to the ones shown in FIG. 5. Such an orthogonal set of capillaries can be created in a place above or below the plane of the ones shown in FIG. 5 so as to avoid intersection between the two sets of orthogonal capillaries.

The present invention can be used to culture embryonic stem cells (ESCs) or induced pluripotent stem cells (iPS) for deriving human tissues of interest for research or for regenerative medicine purposes.

While the ability of ESCs/iPS to generate any type of tissue is well recognized, the actual task of producing tissues of interest using current techniques can be difficult due the lack of a normal regulative environment that coordinates the emergence of defined tissues. In the absence of such an environment, the cells typically differentiate into a mixture of cell types that reflects the arbitrary conditions of the culture. Moreover, without a functional in-vivo-like setting, it is very hard to evaluate the differentiation outcome of human ESCs/iPS-derived cells.

The advantage of the present invention over current approaches relies on the ability to mimic early development in an ex-vivo environment. This capability allows the generation of defined structures and tissues that can not be obtained or assessed by currently available approaches for cell derivation from human pluripotent cells (hPCs). The present system can also include animal embryos as ex-vivo hosts for expanding and assessing the fate of specific populations of cells that are derived from human pluripotent and stem cells and subsequently incorporated into, or adjacently to, the embryos in the niche. While in-vivo incorporation of hPC-derived cells with early animal embryos is technically possible, it is strictly forbidden for ethical reasons due to the possibility of contribution of human cells to the animal germline which could lead to generation of human-animal chimera. This concern is completely alleviated by using the present system to mimic embryonic development. The present system can also be used for differential labeling of cells of different origin or differentiation process and thus enables tracking, analysis and recovery of any desired cellular component (e.g. to re-isolate hPC-derived cells that were injected to a differentially labeled embryo and left to develop within the niche). Note that the use of an embryonic niche is not restricted to embryonic cells, pluripotent cells and embryos. Thus, for example, embryos and/or hPC-derived cells can be injected into a niche that was pre-coated with receptive and/or non-receptive endometrial cell layers. The latter are applied to the niche either in suspension or in a polymerizable (potentially biodegradable) gel or scaffold (e.g., ECM gel, alginate-based scaffold, and other substances known in the field). In an alternative configuration, the embryo or hPC-derived cells are themselves coated with endometrial cells, or co-suspended with these cells, or embedded with these cells in a polymerizable gel. The entire mixture is then applied to the niche.

The present invention can also be used to model the implantation of a human embryo as a way of investigating and improving human fertility. Implantation of the embryo in the uterus occurs early in pregnancy and is influenced by embryonic and maternal factors. While a variety of fertility problems have been overcome using assisted reproductive technologies, implantation failures remain the rate-limiting step for achieving a successful pregnancy. An embryonic niche constructed in accordance with the teachings of the present invention can address a need for studying embryo implantation failure and used to modify the selection of IVF embryos accordingly. It may even be possible to use it for selecting embryos with higher chances of implantation and establishing pregnancies with these embryos. In addition, it can be used to analyze the effects of signaling cues (e.g. natural or synthetic hormones, growth factors, morphogens, etc.), nutrients, gases, and drugs on the progression and efficacy of implantation.

In addition, the present system can be used to study embryo-uterus interactions and their effects on the efficacy of implantation. In such case, the ability to assemble selected components in the present niche enables focused evaluation of the effect and contribution of each component.

For example, the endometrial cells surrounding the embryo or the embryoid body can be selected to be primary cells or cell lines with desired characteristics or desired combinations of characteristics (e.g. a desired balance between receptive and non-receptive endometrium or between epithelial and mesenchymal cells, etc.). Similarly, hPC-derived cells that form the embryoid body can be isolated or manipulated so as to carry specific genetic or epigenetic modifications known or suspected to affect the implantation process (e.g. the use of embryonic stem cells carrying translocations that correlate with implantation failures).

Likewise, the present niche system can be used in conjunction with human embryos carrying specific genetic or epigenetic modifications that may affect the efficacy of implantation, and the associated phenotypes, such as the organization of the surrounding endometrium (e.g. its decidualization, the respective organization of epithelial and mesenchymal tissues, secretion of inflammatory cytokines, etc.), the generation and release of pregnancy hormones (e.g. β-hCG, progesterone, estrogen, etc.), vasculogenesis within the embryonic tissue and its surroundings, etc.

For example, a human blastocyst or a 3D, hPC-based mimic thereof (e.g., hESC-derived embryoid body, EB) can be implanted into an endometrial cell layer within a defined niche environment that provides nutrients and signals resembling those present in-vivo. The embryo or EB will contact the endometrial cells and the mutual organization of the EB and the endometrial cells in the niche can be analyzed in real time. The phenotypic characteristic of the modified epithelial cells include the expression of steroid hormone receptors (estrogen, progesterone, and androgen), luteinizing hormone (LH) receptor and human chorionic gonadotropin (hCG). Specific chemokines and cytokines such as cytokeratin as well as adhesion molecules (mucin 1 and osteopontin) are also expressed and secreted by the receptive endometrial cells. The expression of the above mentioned molecules will be analyzed by real-time PCR (RNA) and Elisa (protein). Human trophoblast growth and migration can be clearly visualized by conventional microscopy.

Molecular markers in the embryo/EBs can be used as complementary means to evaluate trophoblast differentiation. These markers include hCG, progesterone and estrogen [Gerami-Maini et al. 2004]. In case the EB fails to interact with the endometrial cells (e.g., does not spread into the endometrial layer, does not express the above mentioned markers, and/or fails to modify it) the embryo/EB can be coated with trophectodermal cell lines (e.g., Jeg-3, and Jar [White et al. 1988]) and re-evaluated within the niche. The embryo/EB and the endometrial and trophectodermal cells can be applied to the niche in suspension (e.g., in human blastocyst medium such as MultiBlast™, Irvine Scientific) or in a polymerizable (potentially biodegradable) gel or scaffold. The latter can also be supplemented with human blastocyst medium which mimics the uterus fluid and is currently used to culture human blastocysts during IVF. The medium can also be supplemented with various concentrations of progesterone and estrogen, previously shown to be correlated with implantation [Hannan et al. 2010]. This setup can be used in combination with different oxygen concentrations, typically (but not exclusively) ranging from 0% to 21%. Once in the niche, the setup can be exposed to morphogen gradients that are known to polarize the early mouse epiblast, like BMP4 and Wnt3A at one edge of the EB and FGF2 and Nodal at the opposing edge [Beddington and Robertson 1999; Tam et al. 2007].

Moreover, the present niche system allows further control and modification of the gaseous environment in the niche via the same fluid pathways that control diffusion of the nutrients and signals (feeding capillaries). This can be done by maintaining the chamber surrounding the niche at a first gaseous state while maintaining a second gaseous state within the niche through the fluid pathways.

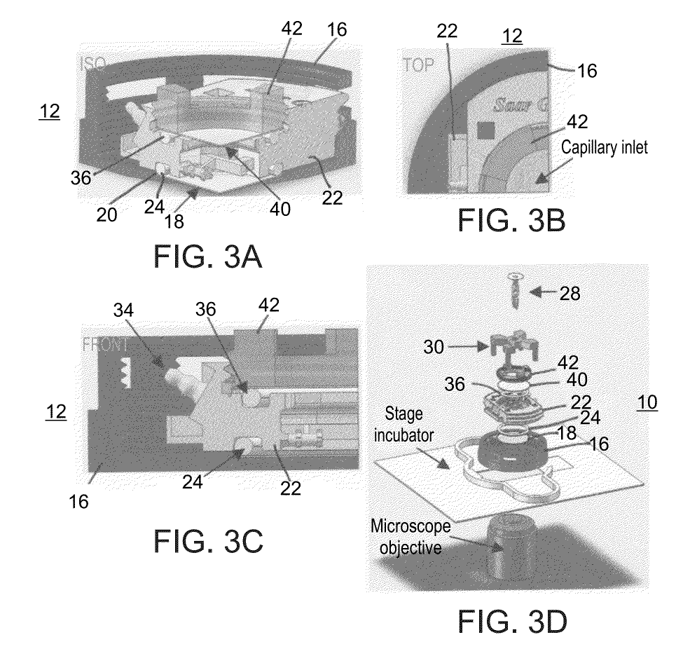

The most attractive readout for the progression of implantation is the secretion level of β-hCG (human chorionic gonadotropin beta), which in fact serves as the gold-standard in pregnancy tests. The levels of β-hCG. (and any other metabolite) can be measured in the outflux of the fluid pathways using any of the standard methods for its detection. This allows continuous, online, non-interfering measurement of the implantation progression. Implantation and development within the niche can further be correlated with additional readouts, such as the reorganization of the endometrium around the embryo/EB, polarization and invagination of the embryo/EB, and the formation of vasculature as determined by morphology (see FIGS. 4A-D). Moreover, following the development in the niche, the embryo/EB is retrieved for high resolution assessment of implantation parameters and embryonic outcome (e.g., type of tissues formed and their polarization within the embryo/EB). This can be done by any standard method such as histology, immunohistochemistry and quantitative real-time PCR. Note that the ability to correlate secretion levels of β-hCG or other materials with functional stages in the implantation is not trivial. This option is not easily available in in-vivo models.

The origin and relative organization of each of the formed tissues can be determined by working with differentially labeled cellular components. For example, the endometrial cells can be stably labeled with a constitutively expressed GFP delivered by standard methods such as viral infection or transfection with a transposable element (e.g., piggyBac™). The EB can be similarly labeled with RFP. A third component, e.g. a trophectodermal cell line can be labeled with CFP or another, non-overlapping fluorescent protein (e.g., e2-Crimson). This allows continuous differentiation between tissues that are derived from these three different sources.

As controls for the effect of signaling gradients on the implantation and development of the embryo/EB, the present system can include two more niches, each juxtaposed to one of the feeding capillaries (FIGS. 1A and D). These niches can be exposed to uniform (but potentially time varying) conditions determined by the respective fluid pathway (FIG. 1D).

The present invention can also be used as a platform for maturation of human Oocytes and/or blastocysts under conditions that more closely mimic the in-vivo environment. A human Oocyte can be placed within the niche with cumulus and granulose cells as a model of the follicle microenvironment. In this case, the temporal and concentration/gradient effects of components such as cyclic AMP (cAMP), luteinizing hormone (LH) and follicle-stimulating hormone (FSH) can be studied and directly correlated with the Oocyte maturation. The key advantage of this configuration over existing approaches is the ability to directly control the diffusion processes around the oocyte that are known to be essential in its development; the diffusion motion of these compounds through gap junctions between the follicle supporting/somatic cells and the oocyte are critical to the maturation. In this case, materials can be tagged fluorescently (e.g., by Fluorescein) and the effects of their motion/concentration within the follicle can be studied by techniques such as fluorescence resonance energy transfer (FRET) or fluorescence recovery after photobleaching (FRAP).

The present invention can also be used as a platform for cancer research and therapy. The flexible and versatile design of the present system enables growth of human tumors within human tissues, thus offering an ex-vivo, human-like environment for analyzing tumorogenesis, and enabling diagnoses of anti-cancer drugs in an environment that complements animal models.

A sample of tumor tissue (and/or organ) of any specific type or stage can be placed in the niche with or without surrounding normal and/or abnormal tissue (and/or organ) of any specific type and composition. The tumor tissue can be of one type or a combination of several types. The types of tumor include, for example, solid, non-solid, primary, cell line, malignant, benign, local, metastasizing, melanoma, carcinoma, adenocarcinoma, sarcoma, lymphoma, glioma or leiomyosarcoma. It can be extracted directly from patients (with or without surrounding tissues) prior to, or following anti-cancer treatment. It can also be a tissue comprising adherent and/or suspension cells that have been grown in culture for any period of time and that were exposed to any type of treatment. The surrounding tissue can be, for example, adherent, non-adherent, stroma, epithelial, neural, glial, connective tissue, endothelial, hematopoietic or any other type of tissue derived from endoderm, mesoderm, ectoderm, and germline.

The tumor and/or the surrounding tissue can be labeled or differentially-labeled with any type of stain and/or live or end point markers. These may or may not indicate the state, stage, identity of the respective tissues. For example, the tumor and the surrounding tissues can be constitutively marked with different fluorescent proteins that distinguish the two tissues. Alternatively, a live cell marker can be expressed in a tissue-specific manner and used to report the differentiation stage and/or state of the cells from each tissue or the marker can be used to indicate the progression of the tumor and/or the viability or health of the cells. Still alternatively, the markers can be used to indicate the types of tissues that are formed (e.g. by pentachrome marking) or to indicate physiological parameters of the tissue such as (but not limited to) the pH, reactive oxygen species, oxygen levels, and the like. Secretion products of the tumor and/or surrounding tissues can be recovered directly from the niche and/or collected through the external ports connected to the fluid pathways. These products can be also be used as markers for the progression and physiology of the tumor and/or surrounding tissue as well as for success of treatment and for the interaction between the tissues.

The tumor and normal tissue can be co-embedded within a biodegradable and/or biocompatible scaffold in 3D. Alternatively, one of the tissues (e.g. the tumor or the surrounding tissue) can be introduced first (with or without a scaffold) with the other tissue subsequently added (with or without a scaffold). Alternatively, either the tumor or the normal tissue is applied within a scaffold while the other tissue is applied as a suspension. The tumor and/or surrounding tissue can include exogenous molecules such as, transgenes, drugs, antibodies, small molecules and the like.

The tumor and/or surrounding tissue can be cultured in the niche for extended periods of time (at least over few weeks) in an environment that is controlled for temperature and gases. In addition, these tissues can be subjected (throughout the culturing period) to a dynamically-controlled gradient factors determined by the user and provided by natural and/or synthetic molecules or cells. These factors include, but are not limited to, potential anti-cancer drugs, growth factors, plasma from patients that may have underwent treatments, cells and the like. The factors can be added directly to the niche or delivered via the fluid pathways. The latter delivery approach further allows full control over the absolute concentration of each factor in the gradient, and separate control over the slope (time based concentrations). Any specific setting can involve simultaneous, factor-specific gradients, each determined by the endpoint, potentially time varying, concentration of the respective factor. The concentration and slope of each factor can be changed over time or the dosage of factors can be varied over time. Alternatively, one set of factors can be replaced (gradually or abruptly) by another set of factors (potentially in response to real-time information derived from morphological and other markers that are being used to assess the state of the tissues in the niche).

The control niches (FIG. 1A) can be used in parallel with the gradient niche so as to determine the effect of the gradient compared with the effect of the end point concentrations of the gradients. The factors that are delivered through the fluid pathways can be re-circulated therethrough. For example, cells that are reactive or that could become reactive to the tumor (e.g. by any of the known and yet to be discovered methods of adaptive cellular immunotherapy) can be re-circulated through the fluid pathways so as to make such cells progressively more reactive to the tumor. This enables expansion of tumor-reactive cell populations. The tissues within the niche can include tumor reactive cells (e.g. immune cells) that expand and become reactive to the tumor following the circulation of specific tumor antigens that have the potential of activating the immune system against the tumor.

Throughout the incubation period, the entire setup can be visualized by time lapse microscopy, e.g. confocal fluorescence microscopy. The system can also be placed within a tissue culture incubator and monitored with or without additional biochemical and cellular factors.

the present system can also be used to monitor the effects of potential drugs and/or physiological fluids (such as but not limited to plasma from treated patients or immunized cells from patients) and/or potentially tumor reactive cells on tumor tissue using, for example, an array of niches (such as that shown in FIG. 5). Potential drugs/cells can be applied either through the fluid pathways or directly into the niche. A type of surrounding tissue can be tested in parallel by having different niches accommodated by different combination of tumor and surrounding tissues. Analysis of outcomes can be performed in post culturing or in real time by using scanning devices. This analysis can incorporate any type of marker used or known in the field and allows rapid identification of biochemical and/or cellular factors that can shrink, compromise, eradicate, and/or change the tumor in any way. Such scanning can also be used to identify factors that adversely affect the accompanying normal tissue or that support the health of the normal tissue. Alternatively, scanning can be used to identify factors with the inverse activity of advancing the tumor and/or deteriorating the normal tissue (tumorigenic factors). The system can also be used to identify and evaluate side effects of each treatment on the accompanying normal tissue. Factors recovered from the tumor and/or the normal tissue (either directly or via the outlet port) can be analyzed (biochemically or molecularly), and correlated with the state, stage, and any parameter of the tumor so as to enable construction of a prognostic database.

Additional applications of the present invention include, but are not limited to:

(i) Neurogenesis in dynamically controlled 3D environments (e.g. identifying the identity, composition, concentration, arrangement, and duration of application of biochemical and/or cellular factors affecting neurogenesis);

(ii) Angiogenesis and vasculogenesis in dynamically controlled 3D environments (e.g. identifying the identity, composition, concentration, arrangement, and duration of application of biochemical and/or cellular factors affecting angiogenesis and vasculogenesis);

(iii) Tissue regeneration in dynamically controlled 3D environments (e.g. identifying the identity, composition, concentration, arrangement, and duration of application of biochemical and/or cellular factors affecting tissue regeneration);

(iv) Chemotaxis/migration assays in a 3D environment;

(v) Screening for apoptotic factors; and

(vi) Screening of new biodegradable scaffolds.

Thus, the present invention provides a cell culturing chamber and system which more closely mimic the conditions of in-vivo niches and thus enables culturing of stem cells, EBs (embryoid bodies) and embryos in a controllable manner. The present system supports dynamic control of the spatio-temporal concentrations and gradients of the biochemical components (e.g., morphogens, nutrients and soluble gases) all throughout the entire culturing period. It also contains tools for generating the niche and injecting the embryo/EB into the niche. The system is inherently accessible for live-cell time lapse confocal microscopy over an extended period of time, online collection of metabolites, and sample retrieval for analysis at the end of the experiment. Fixation and immunohistochemistry of samples can also be effected via perfusion of fixation and immunohistochemistry reagents through the culturing chamber.

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Example 1

### Diffusion Gradients

The present system was tested with small and large molecules in order to determine the diffusion gradients for these molecules.

The Food colorants Green-mix (mixture of E133/Brilliant Blue FCF, M_{w}=792.84 Da, and E110/Sunset Yellow FCF, M_{w}=452.37 Da) and Red-Azorubine (E122, M_{w}=502.4 Da) and the fluorescent molecule Calcein (Sigma-Aldrich C0875, Mw=622.53 Da) were used as models for small molecules (FIGS. 2N-R). The kinetics and spatial distribution of the Calcein was investigated using confocal microscopy time lapse (Zeiss LSM 710) and is presented in FIG. 2R. In this Figure, a simple moving average (SMA, fifty elements) data smoothing algorithm is used to filter the noise. FIG. 2R demonstrates the chamber ability to maintain a stable small molecule gradient over an extended period of hours. This Figure also illusatrtes that the gradient rapidly stabilizes within less than two hours.

To demonstrate the ability of the present system to maintain a stable gradient of proteins over long periods of time a model GFP-coh2 fusion protein (M_{w}˜46.5 kDa) was employed. This protein is both fluorescent and represents a good model for morphogens such as BMP-4 kDa).

Briefly, a His-tagged (C-terminus) GFP-coh2 fusion protein (M_{w}˜46.5 kDa) was expressed and batch-purified by Ni-NTA affinity chromatography, essentially as described previously (Karpol et al. 2008, Vazana et al. 2010, Demishtein et al. 2010). E. coli BL21(λDE3) cells were transformed with pET28_GFP-coh2, kindly provided by Prof. Edward A. Bayer, and grown at 37° C. in Luria-Bertani broth supplemented with 50 μg/ml kanamycin to an OD₆₀₀ of ˜1. Isopropyl-1-thio-β-D-galactoside (IPTG) was added to a final concentration of 0.2 mM, for induction of protein expression, and the culture further grown for 3 hours. Cells were harvested by centrifugation (5,000 g, 15 min, 4° C.) and resuspended in Tris-buffered saline (TBS, 137 mM NaCl, 2.7 mM KCl, 25 mM Tris-HCl, pH 7.4; 20 ml TBS per 500 ml culture harvest) supplemented with 5 mM imidazole and protease inhibitor (1 mM phenylmethylsulfonyl fluoride [PMSF], 0.4 mM benzamidine, and 0.06 mM benzamide) and lysed by sonication (on ice). The lysate was heated for 30 min at 52° C. and centrifuged (20,000 g, 30 min, 4° C.). The supernatant fluids were mixed with Ni-nitrilotriacetic acid (NTA) supplemented with 5 mM imidazole and rotated (12 RPM) for 45 minutes at 4° C. A Ni-NTA column (AKTA-prime System, GEHealthcare, Pittsburgh, Pa.) was pre-washed (gravity flow) with five column volumes of the resuspension buffer. Protein elution was performed by using TBS supplemented with 20, 100, 250 and 500 mM imidazole. Fractions (2 ml) were collected from each imidazole concentration group until GFP ceased eluting (visual assessment), analyzed on SDS-PAGE (10%) and visualized by Coomassie brilliant blue (CBB) staining. The fractions containing sufficiently purified protein were pooled, aliquoted, flash frozen and stored at −80° C. The protein concentration was estimated by absorbance at 280 nm. The extinction coefficient (44810 cm⁻¹M⁻¹) was determined using ProtParam (www.expasy.org/tools/protparam.html) (Gasteiger et al. 2003, 2005). FIG. 2S presents the GFP-Coh2 kinetics and distribution in the chamber during 27 hours of perfusion. This Figure illustrates the ability of the present system to maintain a stable protein gradient over an extended period of hours. In contrast to the small molecule levels that stabilize within two hours, the protein levels climb only after about five hours. This is expected due to its larger molecular weight and its reduced diffusivity.

### Example 2

### Culturing of EBs

Several cell lines (293T, ECC-1, Ishikawa, JAR, H1/H9/Hues hESCs), fruit fly embryos (D. melanogaster) and EBs (hESCs, constitutively labeled with CFP or genetically marked for the expression of the pluripotency gene OCT4 with GFP—a knock-in line kindly provided by the Thomson lab, FIG. 4D) were used in order to demonstrate the ability of the present system to support culturing of embryonic models. The present system was tested for the ability to induce organization of EBs in a gradient niche (vs. control niches). The effects of in vivo-like signals (e.g., BMP-4 and FGF) on the polarization were tested. The simple readout for these experiments is spatially organized differentiation responses within the EBs.

Two approaches were tested for establishment of an implantation model. The first approach relied on generating human EBs consisting of both trophoblast and ESC lines and co-culturing them with receptive endometrial lines (FIG. 4C). The second approach used bovine and mouse blastocysts with primary endometrium (derived from the host organism, FIG. 4A).

Results from the first approach indicated that it is advantageous in terms of control over the genotypic characteristics of the cells (e.g., constitutive labels), while results from the second approach indicated that it is advantageous in terms of biological authenticity (mimicking true embryo).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES

[1] Gilbert S F (2006) Developmental Biology, Eighth Edition, Sinauer Associates, Sunderland, Mass., USA.
[2] King J A, Miller W M (2007) Bioreactor development for stem cell expansion and controlled differentiation, Current Opinion in Chemical Biology 11:394-398
[3] Li L, Xie T (2005) Stem cell niche: structure and function, Annual Review of Cell and Developmental Biology 21: 605-631
[4] Mo B, Vendrov A E, Palomino W A, DuPont B R, Apparao K B C, Lessey B A (2006) ECC-1 cells: A well-differentiated steroid-responsive endometrial cell line with characteristics of luminal epithelium, Biology of reproduction 75: 387-94.
[5] Pawley J B (1995) Handbook of biological confocal microscopy, Second Edition, Springer, NY, N.Y., USA.
[6] Sahal K, Pollock J F, Schaffer D V, Healy K E (2007) Designing synthetic materials to control stem cell phenotype, Current Opinion in Chemical Biology 11:381-387
[7] Scadden D T (2006) The stem-cell niche as an entity of action, Nature 441: 1075-1079
[8] Thomson J A, Itskovitz-Eldor J, Shapiro S S, Waknitz M A, Swiergiel J J, Marshall V S, Jones J M (1998) Embryonic stem cell lines derived from human blastocysts. Science 282: 1145-7.
[9] Wang H, Dey S K (2006) Roadmap to embryo implantation: clues from mouse models, Nature reviews genetics 7: 185-99
[10] Won Choi N, Cabodi M, Held B, Gleghorn J P, Bonassar L J, Stroock A D (2007) Microfluidic scaffolds for tissue engineering, Nature Materials 6: 908-15.
[11] Janaky Narayanan, Jun-Ying Xiong, Xiang-Yang Liu (2006) Journal of Physics—Conference Series 28: 83-86.
[12] Gerami-Naini, B., et al., Trophoblast differentiation in embryoid bodies derived from human embryonic stem cells. Endocrinology, 2004. 145(4): p. 1517-24.
[13] White, T. E., et al., Human choriocarcinoma (JAr) cells grown as multicellular spheroids. Placenta, 1988. 9(6): p. 583-98.
[14] Hannan, N. J., et al., Models for study of human embryo implantation: choice of cell lines? Biol Reprod, 2010. 82(2): p. 235-45.
[15] Beddington, R S, and Robertson (1999), E J. Axis development and early asymmetry in mammals. Cell 96:195-209.
[16] Tam, P P, and Loebel, D A. (2007) Gene function in mouse embryogenesis: get set for gastrulation. Nat Rev Genet 8:368-81.

What is claimed is:

1. A cell culturing system comprising at least one cell niche defined by a niche base and niche walls, wherein at least one niche wall of said niche walls includes a fluid pathway formed within said niche wall and further wherein a material of said niche wall is selected capable of enabling diffusion into said at least one cell niche of a fluid flowing between an inlet and an outlet of said fluid pathway.

2. The cell culturing system of claim 1, wherein said material is a biopolymer gel.

3. The cell culturing system of claim 1, wherein said niche walls angle out from said niche base.

4. The cell culturing system of claim 1, further comprising a support scaffold for supporting at least one cell niche.

5. The cell culturing system of claim 1, further comprising a biodegradable scaffold contained within said cell niche.

6. The cell culturing system of claim 5, further comprising biomolecules and/or cells trapped within or attached to said biodegradable scaffold.

7. The cell culturing system of claim 1, wherein said material enables diffusion of molecules carried by said fluid.

8. The cell culturing system of claim 1, wherein said material is impermeable to diffusion of cells.

9. The cell culturing system of claim 4, further comprising a chamber for housing said support scaffold.

10. The cell culturing system of claim 9, wherein said chamber includes a bottom cover slip mountable on a microscope stage.

11. A method of fabricating a cell culturing chamber comprising:
(a) 3D printing a housing of the cell culturing chamber, said housing having inlet and outlet ports and a support scaffold for containing at least one cell culturing niche; and
(b) casting said at least cell culturing niche in said support scaffold.

12. The method of claim 11, wherein said housing includes an internal component threadable into an external component.

13. The method of claim 11, wherein said at least one cell culturing niche is cast from a biopolymer gel.

14. The method of claim 11, wherein said cell culturing niche is cast over at least one wire positioned between said inlet and said outlet ports, said wire being removed following casting to form a fluid pathway in a wall of said at least one culturing niche.

15. The method of claim 11, wherein said housing further comprises top and bottom cover slips.

16. A method of culturing cells or cell structures comprising seeding cells or cell structures into the culturing system of claim 1.

17. The method of claim 16, wherein said cells or cell structures are seeded with a biodegradable scaffold.
